(19) United States
(12) Patent Application Publication
(10) Pub. No.: US 2006/0290882 A1
(43) Pub. Date: Dec. 28, 2006

(54) LAMINATED CONTACT LENS

(75) Inventors: William E. Meyers, Scottsdale, AZ (US); Hank L. Stute, Queen Creek, AZ (US)

Correspondence Address: SNELL & WILMER, 400 EAST VAN BUREN, ONE ARIZONA CENTER, PHOENIX, AZ, 85004-2202, US

(73) Assignee: PARAGON VISION SCIENCES, INC., Mesa, AZ (US)

(21) Appl. No.: 11/425,827

(22) Filed: Jun. 22, 2006

Related U.S. Application Data
(60) Provisional application No. 60/595,370, filed on Jun. 27, 2005.

(51) Int. Cl. G02C 7/04; A61K 9/00

(52) U.S. Cl. 351/160 H; 424/429

(57) ABSTRACT

A laminated contact lens according to the present invention includes at least a first contact lens and a second contact lens coupled to each other via the peripheral edge of each respective contact lens. The two contact lenses include a proximity zone depth and/or proximity zone width different from one another such that one or more chambers are formed between the two contact lenses. Various materials and/or components may be housed within the chamber to provide, among other things, vision correction tools, medicament administration, and/or cosmetic improvements to the user.

### CROSS-REFERENCE TO RELATED APPLICATIONS

This application claims priority to, and the benefit of, U.S. Provisional Application Ser. No. 60/595,370 entitled “Laminated Contact Lens,” filed on Jun. 27, 2005, and hereby incorporates such provisional application by reference, in its entirety.

### FIELD OF INVENTION

The invention generally relates to contact lenses, and particularly to, a laminated contact lens having one or more chambers formed between two or more contact lens layers.

### BACKGROUND OF THE INVENTION

Contact lenses have existed since the time of Leonardo Da Vinci. Historically, contact lenses have been used to correct the vision of the user. In the early to mid twentieth century, contact lenses were beginning to be formed of plastic materials rather than glass. In the mid to late twentieth century, contact lens began to be formed of more flexible plastic materials.

Typical contact lenses include a circular or elliptical shape formed of a single contact lens. Since contact lenses typically include only a single layer, the contact lens is generally capable of performing a limited number of functions. For example, the primary use of contact lenses continues to be vision correction. However, some contact lenses have also been used to manipulate the shape of the cornea such that improved vision is achieved without needing to continuously wear the contact lens. Furthermore, some contact lenses include a permanent color such that the user appears to have different color eyes when wearing the contact lens.

While different uses for contact lenses exist, the fact that typical contact lenses include a single continuous layer limits the utility of the contact lens. Should a manufacturer wish to make a lens with a function different than a contact lens the manufacturer is currently producing, the manufacturer is usually required to make substantial changes to the design and/or manufacturing process. As such, the cost of making contact lenses for different functions is often costly. Therefore, a contact lens which allows a manufacturer to make a base contact lens with the ability to perform multiple functions, and including new uses, with relatively minor alterations is needed in the art.

### SUMMARY OF THE INVENTION

A contact lens according to various exemplary embodiments of the invention is an ocular prosthesis, capable of acting as a contact lens and a device simultaneously capable of other desirable functions, deriving from the inclusion of accessible chambers within the prosthesis, the formation of the chambers being such that they remain suitably accessible for placement of substances, partitions or devices, having secondary utility, after the completion of manufacture of the enclosing lens body, the chambers therein being either secluded from or in communication with the fluids and surfaces of the eye.

For example, in accordance with various embodiments of the present invention, contact lens includes a first contact lens including a peripheral edge, a first variable proximity zone depth (PZD), and a first variable proximity zone width (PZW); and a second contact lens including a peripheral edge, a second variable PZD, and a second variable PZW, wherein the peripheral edge of the first contact lens is coupled to the peripheral edge of the second contact lens, the first variable PZD is different than the second variable PZD, and the first variable PZW is different than the second variable PZW.

Momentarily, “edge” is used often herein, and when used should be appreciated as designating a continuous surface.

In accordance with an aspect of one exemplary embodiment of the invention, the difference between the first variable PZD and the second variable PZD is in the range of about 10 microns to about 1000 microns. In another aspect of the invention, the difference between the first variable PZD and the second variable PZD is about 200 microns. In yet another aspect of the invention, the difference between the first variable PZW and the second variable PZW is in the range of about 250 microns to about 5500 microns. In still another aspect of the invention, the difference between the first variable PZW and the second variable PZW is about 5250 microns.

In an exemplary embodiment, the difference between the first variable PZD and the second variable PZD, and/or the first variable PZW and the second variable PZW creates one or more chambers between the first contact lens and the second contact lens. The volume of each chamber is capable of being manipulated by the size of the contact lens, the absolute and relative widths and/or radial locations of the corresponding zones of the anterior and posterior lenses, the difference between the first variable PZD and the second variable PZD, and/or the difference between the first variable PZW and the second variable PZW of the first contact lens and the second contact lens, respectively. The formation of the chamber enables different materials and/or components having functions differing from one other to be housed by the chamber. As such, by changing/interchanging the different materials and/or components that are housed by the chamber, the function(s) of the contact lens may be changed.

The geometry of the four central surfaces of the lens may be manipulated to establish the optical (e.g., refractive or diffractive) properties of the lens and/or create a plurality of chambers. In addition, the composition of the materials of the anterior and posterior lens may be different from one another to alter the function of the lens.

### BRIEF DESCRIPTION OF THE DRAWINGS

A more complete understanding of the present invention may be derived by referring to the detailed description when considered in connection with the Figures, where like reference numbers refer to similar elements throughout the Figures, and

FIG. 1 is a diagram of one exemplary embodiment of a laminated contact lens formed of two or more contact lenses coupled to each other and forming a chamber between at least two of the contact lenses;

FIG. 2 is a diagram of the laminated contact lens illustrated in FIG. 1 having a material and/or component housed within the chamber;

FIG. 3 is a diagram of another exemplary embodiment of a laminated contact lens formed of two or more contact lenses coupled to each other and forming a chamber between at least two of the contact lenses and including a bonding composition capable of transporting one or more materials through the bonding composition;

FIG. 4 is a diagram of yet another exemplary embodiment of a laminated contact lens formed of two or more contact lenses coupled to each other and forming a chamber between at least two of the contact lenses, wherein the two or more contact lenses contact each other at a peripheral edge of each respective lens, and at a central point of each respective lens;

FIGS. 5-20 and 22-23 are diagrams illustrating exemplary embodiments of various laminated contact lenses formed of two or more contact lenses coupled to each other, wherein each laminated contact lens includes a plurality of chambers between at least two of the contact lenses; and

FIG. 21 is block diagram of a laminated contact lens made in accordance with one embodiment of a method for making a laminated contact lens.

### DETAILED DESCRIPTION

The detailed description of exemplary embodiments of the invention herein makes reference to the accompanying Figures, which show the exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation.

For the sake of brevity, functional embodiments of the apparatus and systems (and components of the individual operating components of the systems) may not be described in detail herein. Furthermore, the connecting lines shown in the various figures contained herein are intended to represent exemplary functional relationships and/or physical connection between the various elements. It should be noted that many alternative or additional functional relationships or physical connections may be present in a practical system.

Embodiments of the present invention provide a laminated contact lens having a chamber formed between two or more contact lenses. The laminated contact lens enables a manufacturer to make several different types of contact lenses from a base contact lens with relatively minor alterations. Furthermore, various exemplary embodiments of the invention provide uses for contact lenses that have heretofore been unavailable.

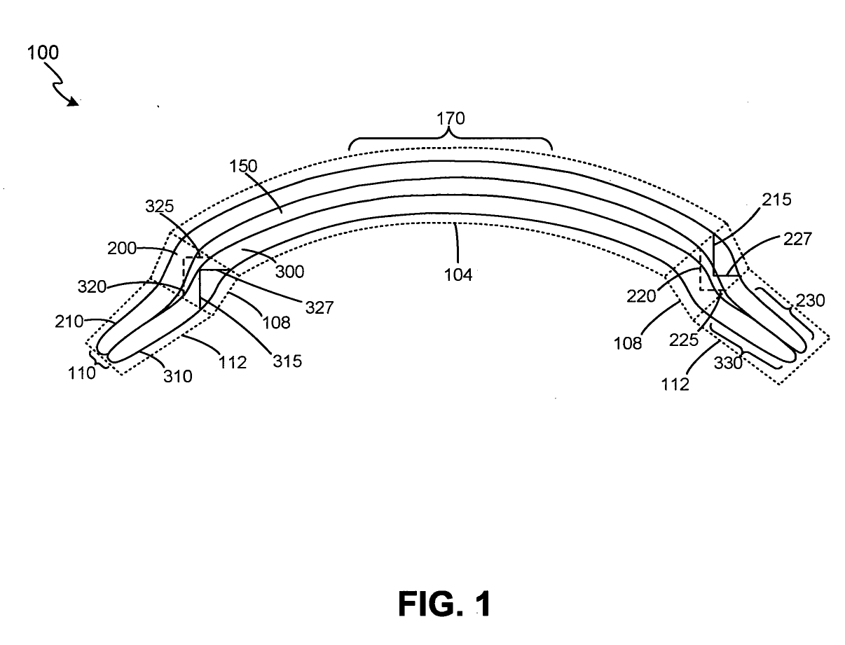

Turning now to the Figures, FIG. 1 illustrates a diagram of one exemplary embodiment of a laminated contact lens 100. Laminated contact lens 100, in an exemplary embodiment, is formed by coupling a first contact lens 200 having a peripheral edge 210, a variable proximity zone depths (PZD) 215, 220, (anterior and posterior), and variable proximity zone widths (PZW) 225, 227 (posterior and anterior) to a second contact lens 300 having a peripheral edge 310, a variable PZD 315, 320 (posterior and anterior), and a PZW 325, 327 (anterior and posterior) wherein the chord diameter of laminated contact lens 100 is in the range of about 8.0 millimeters (mm) to about 16.0 mm (i.e., a semi-chord diameter in the range of about 4.0 mm to about 8.0 mm). Contact lens 200 and/or contact lens 300 may be any contact lens capable of being suitably configured according to any and/or all of the exemplary embodiments discussed below. Furthermore, in various exemplary embodiments, contact lens 200 and/or contact lens 300 is a contact lens manufactured by Paragon Vision Science of Mesa, Ariz. under the name Paragon CRT®. Moreover, other designs capable of altering/modifying the sagittal depth of a contact lens such as, for example, “reverse geometry” lenses or lenses with peripheral curves originating from points not on the central axis of the central zones are also contemplated by the invention to create lenses of the present geometry.

In one embodiment, contact lens 200 is coupled to contact lens 300 via peripheral edge 210 and peripheral edge 310 to form a central zone 104, an intermediate zone 108, and a peripheral zone 112 of laminated contact lens 100. In accordance with one exemplary embodiment, the radius of curvature of central zone 104 is in the range of about 6.0 mm to about 10.0 mm. on the posterior side (i.e., the cornea side) and in the range of about 5.0 mm to about 12.0 mm on the anterior side. Furthermore, central zone 104, in one embodiment, includes a corrective zone 170 for correcting the vision of the user. Thus, corrective zone 170 may be appropriately configured according to a particular user's eye dimensions and/or prescription needs, which optical properties are determined by the refractive indices of contact lens 200 and contact lens 300, that of any media (discussed below) filling the gap in corrective zone 170, and the radii of curvature of the posterior and anterior surfaces of contact lens 200 and the radii of curvature of the posterior and anterior surfaces of contact lens 300, each of which radii of curvature may be spherical or aspherical, as desired.

Intermediate zone 108 is formed by PZD 215, 220, PZW 225, 227, PZD 315, 320 and PZW 325, 327 wherein the width along the semi-chord of intermediate zone 108 is in the range of about 0.25 mm to about 5.5 mm, or is a proportion of the full chord diameter in the range of about 1.5 percent to about 35 percent. In accordance with an aspect of one exemplary embodiment of the invention, variable PZD 220 is in the range of about 10 microns to about 1010 microns. In another aspect of the invention, variable PZD 215, 315, 320 is in the range of about 10 microns to about 1010 microns. In one exemplary embodiment, variable PZD 220 includes different dimensions than variable PZD 320 such that a chamber 150 is formed when contact lens 200 is coupled to contact lens 300. In accordance with an aspect of one exemplary embodiment of the invention, the difference between variable PZD 220 and variable PZD 320 is in the range of about 10 microns to about 1010 microns when forming chamber 150. In another aspect of the invention, the difference between variable PZD 220 and variable PZD 320 is about 200 microns when forming chamber 150.

PZW 225, in one embodiment, is in the range of about 250 microns to about 5500 microns. Furthermore, PZW 222, 325, 327, in an embodiment, is in the range of about 250 microns to about 5500 microns. In one exemplary embodiment, variable PZW 225 includes different dimensions than variable PZW 325 such that a chamber 150 is formed when contact lens 200 is coupled to contact lens 300. In accordance with an aspect of one exemplary embodiment of the invention, the difference between variable PZW 225 and variable PZW 325 is in the range of about zero microns to about 5250 microns when forming chamber 150. Thus, chamber 150 may be formed by the difference between PZD 220 and PZD 320 (i.e., modifying the dimensions of the sagittal depth), and/or by the difference between PZW 225 and PZW 325. In accordance with various exemplary embodiments, the various PZD and PZW dimensions may vary in a near infinite number of configurations, though, generally the thickness of individual lenses 200, 300 are within a range of about 0.7 mm and about 0.02 mm, preferably, between about 0.3 mm and about 0.03 mm, and some most preferable embodiments, between about 0.2 mm and about 0.04 mm.

Peripheral zone 112 is formed of peripheral edge 210 and peripheral edge 310, wherein, in one exemplary embodiment, each of peripheral edge 210 and peripheral edge 310 include a flat landing zone 230 and a flat landing zone 330, respectively, and flat landing zones 230 and 330 substantially correspond to one another, though, in alternative embodiments the peripheral edge may be curved, while maintaining correspondence one lens to another as required for coupling.

In one exemplary embodiment, a peripheral edge 110 of laminated contact lens 100 is formed when contact lens 200 is coupled to contact lens 300 via flat landing zones 230 and 330, respectively. In general, peripheral zone 112 includes a width along the semi-chord diameter in the range of about 0.5 mm to about 5.5 mm. In accordance with an aspect of one exemplary embodiment of the invention, the width along the semi-chord diameter is preferably in the range of about 1.0 mm to about 3.0 mm. In accordance with another aspect of the invention, the width along the semi-chord diameter is most preferably in the range of about 1.0 mm to about 2.0 mm. Furthermore, in accordance with one exemplary embodiment, peripheral zone 112 includes a radius of curvature in the range of about 7.0 mm to about 14.0 mm for each of the posterior and anterior sides.

Notably, the invention contemplates that contact lens 200 and/or contact lens 300 may each include a plurality of proximity zones along the semi-chord diameter of each respective contact lens. By configuring the plurality of proximity zones in each respective lens differently and/or independently from one another, chamber 150 may include an infinite number of configurations (e.g., shapes and volumes). Thus, chamber 150 is capable of having an infinite number of a pre-determined shapes and/or volumes.

Furthermore, the invention contemplates that laminated contact lens 100 may be formed of any transparent polymer capable of being utilized in a contact lens, and generally includes a radius in the range of about 4.0 mm to about 8.0 mm. In various exemplary embodiments, laminated contact lens 100 is formed of a polymer and/or copolymer material. In other exemplary embodiments, laminated contact lens 100 is formed of a hydrophilic and/or a hydrophobic monomer. Suitable hydrophilic co-monomers include, but are not limited to, hydroxyl-substituted lower alkyl acrylates and methacrylates, acrylamide, methacrylamide, (lower alkyl) acrylamides and -methacrylamides, ethoxylated acrylates and methacrylates, hydroxyl-substituted (lower alkyl) aclylamides and -methacrylamides, hydroxyl substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone. 2-vinyloxazoline, 2-vinyl-4,4′-diakyloxazoli-5-one. 2- and 4-vinylpyridine, vinylically unsaturated carboxylic acids having a total of 3 to 5 carbon atoms, amino(lower alkyl)-(where the term “amino” also includes quaternary ammonium), mono (lower alkylamino) (lower alkyl) and di (lower alkylamino)(lower alkyl) acrylates and methacrylates, allyl alcohol, and the like. Examples of other suitable hydrophilic co-monomers include, but are not limited to, hydroxyethy methacrylate (HEMA), Hydroxy ethylacrylate hydroxypropyl acrylate, trimethylammonium 2-hydroxy propylmethacrylate hydrochloride dimethylaminoethyl methacrylate (DMAEMA), dimethylaminoethylmethacrylamide, acrylamide methacrylamide, N,N-dimethylacrylamide (DMA), allyl alcohol, vinylpyridine, glycerol methacrylate, N-(1,1-dimethyl-3-oxobutyl) acrylamide, N-vinyl-2-pyrrolidone (NVP), acrylic acid, methacrylic acid and the like.

Suitable hydrophobic co-monomers include, but are not limited to, C₁-C₁₈, alkyl and C₃-C₁₈, cycloalkyl acrylates and methacrylates, C₃-C₁₈ alkylacrylamides and -methacrylamides acrylonitrile, methacrylonitrile, vinyl C₁-C₁₈ alkanoates, C₂-C₁₈ alkenes, C₂-C₁₈ haloalkenes, styrene, (lower alkyl)styrene, lower alkyl vinyl ethers, C₂-C₁₀ perfluoroalkyl acrylates and methacrylates and correspondingly partially fluorinated acrylates and methacrylates, C₃-C₁₂ perfluoroalkylethylthiocarbonylaminoethyl acrylates and methacrylates, acryloxy- and methacryloxyalkylsiloxanes, N-vinylcarbazole, C₁, —C₁₂, alkyl esters of maleic acid, fumaric acid, itaconic acid, mesaconic acid, and the like. Preference is given, for example, to acrylonitrile, C₁—, C₄, alkyl esters of vinylically unsaturated carboxylic acids having 3 to 5 carbon atoms or vinyl esters of carboxylic acids having up to 5 carbon atoms and difunctional acrylic siloxanes. Examples of other suitable hydrophobic co-monomers include, but are not limited to, methyl acrylate, ethyl acrylate, propyl acrylate isopropyl acrylate, cyclohexyl acrylate, 2-ethylhexyl acrylate, methyl methacrylate ethyl methacrylate propyl methacrylate, butyl acrylate, vinyl acetate, vinyl propionate, vinyl butyrate vinyl valerate, styrene, chloroprene, vinyl chloride, vinylidene chloride acrylonitrile, 1-butene, butadiene, methacrylonitrile, vinyltoluene, vinyl ethyl ether, perfluoro hexylethylthiocarbonylaminoethyl methacrylate, isobornyl methacrylate, trifluoroethyl methacrylate hexafluoroisoprpyl methacrylate, hexafluorobutyl methacrylate tristrimethylsilyloxysilylpropyl methacrylate (TRIS), 3.-methacryloxy propylpentamethyldisiloxane and bis(methacryloxypropyl) tetramethyldisiloxane, N-[tris(trimethylsiloxy)-silylpropyl]methacrylamide (“TSMAA”), N-[tris(-trimethylsiloxy) silylpropyl]acrylamide (“TSAA”), and the like.

In one exemplary embodiment, laminated contact lens 100 is configured to be used during the day. In another exemplary embodiment, laminated contact lens 100 is configured to be used at night and/or while sleeping. Contact lens 100, in accordance with an aspect of one exemplary embodiment of the invention, is configured to only be worn while sleeping. In still another exemplary embodiment, laminated contact lens 100 is configured to be used both during the day, at night, and/or while sleeping. Furthermore, laminated contact lens 100 may be configured to correct the visual performance of the eye by direct refraction or diffraction of light or by reshaping the cornea, as in orthokeratology or corneal refractive therapy.

As such, laminated contact lens 100 allows a user to wear laminated contact lens 100 with substantial comfort. Furthermore, the invention contemplates that laminated contact lens 100 may be formed of any material known in the art capable of forming a laminated contact lens. Furthermore, laminated contact lens 100 preferably forms a generally circular or elliptical shape, however, the invention contemplates that laminated contact lens 100 may include any shape and/or other appropriate configuration.

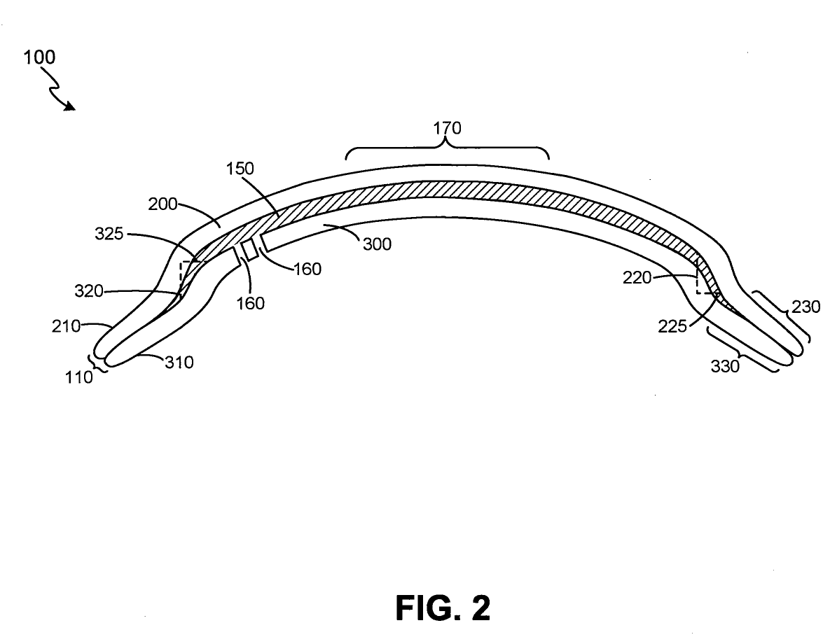

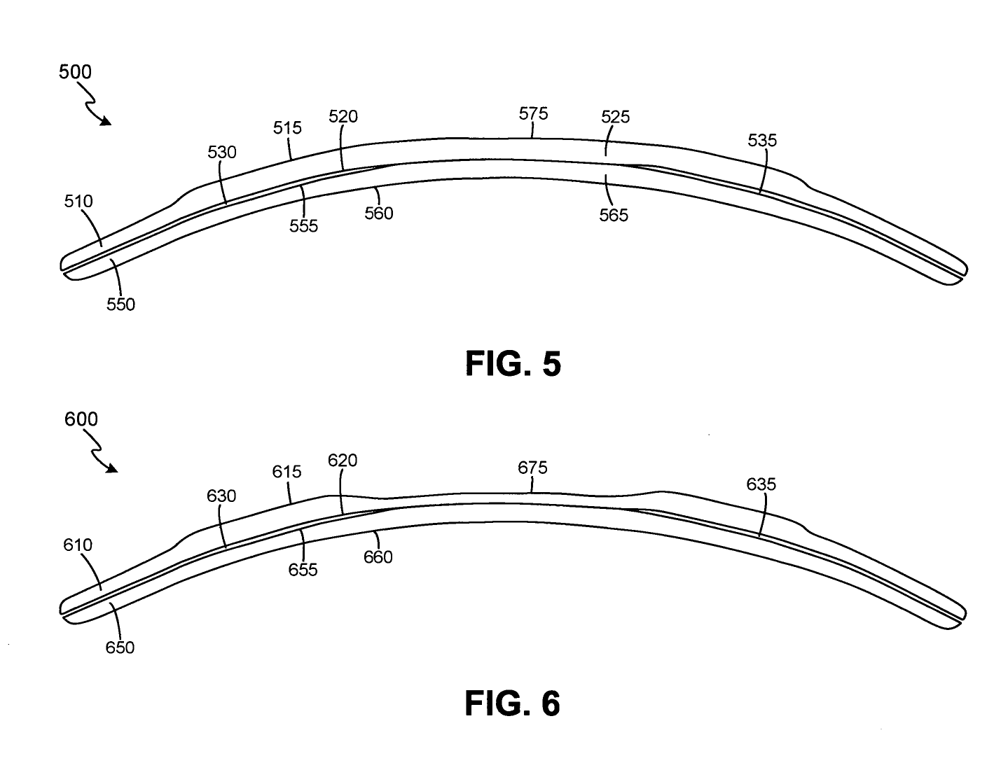

Additionally, the lamination of lenses in accordance with the present invention, creates various edges. For example, FIGS. 1-4 illustrate an invaginated tip, while FIGS. 5-20 and 22 illustrate a semi-rounded tip of the respective edges. As noted above, various embodiments of the invention provide uses for contact lenses that were heretofore unavailable. With reference to FIG. 2, this figure is a diagram of laminated contact lens 100 with a component and/or a material housed within chamber 150 to provide a new use for contact lenses. Chamber 150, in one exemplary embodiment, is filled with a liquid, a gas, and/or a solid. In accordance with various aspects of one exemplary embodiment of the invention, the liquid, gas, and/or solid may be a medicament, a lubricant, and/or any other material suitable for treating a patient (e.g., a human or animal). In another aspect of the invention, contact lens 200 and/or contact lens 300 may be formed of a permeable or semi-permeable material so that at least a portion of the medicament is capable of being delivered to an eye of the user. In yet another aspect of the invention, contact lens 200 and/or contact lens 300 may be configured to deliver the medicament to the eye at a controlled rate by diffusion and/or through holes 160. Holes 160, in various aspects of the invention, may be located in the front and/or back of chamber 150, and/or laterally through peripheral edge 100. In another aspect of the invention, holes 160 are laser-drilled holes suitably configured to enable the liquid, gas, and/or solid in chamber 150 to come in contact with the eye.

In accordance with an aspect of one exemplary embodiment of the invention, the liquid, gas, and/or solid may include a refractive index such that laminated contact lens 100 includes two or more optical zones (not shown). In one aspect of the invention, the two or more optical zones are configured such that chamber 150 forms a multi-focal (e.g., bifocal) optical zone. In another aspect of the invention, the two or more optical zones are configured such that chamber 150 forms a variable power optical zone.

In accordance with another exemplary embodiment, chamber 150 is configured to house one or more color components such that when laminated contact lens 100 is worn, the eye of the user appears to have the color of the color component. For example, when a blue color component is placed within chamber 150, the user, which may have a brown eye, will now appear to have a blue eye. Notably, the invention contemplates that the color components may include any known color and may include any suitable material known in the art capable of being housed in the chamber.

In one aspect of the invention, chamber 150 may be configured such that the color components are replaceable/interchangeable so that the user has the option of replacing/interchanging the color components on a periodic basis such that the user's eyes have the appearance of different colors at different times. In another aspect of the invention, the color components may include light emitting fluorescent and/or phosphorescent components and/or materials wherein the light absorbing profile of which is capable of being externally manipulated.

Laminated contact lens 100, in yet another exemplary embodiment, includes one or more nano-scale mechanical systems housed in chamber 150. In accordance with still another exemplary embodiment, laminated contact lens 150 includes one or more photocells and/or other energy storage devices housed in chamber 150. In accordance with a further exemplary embodiment, laminated contact lens 150 includes at least one nano-scale mechanical system and at least one photocell housed in chamber 150. The nano-scale mechanical system(s) and/or the photocell(s), in one aspect of the invention, may include communication media configured to receive and display images/messages in an appropriate manner that enables the user to view such. For example, the user may be able to view text messages, pictures, video streams, and/or other viewable material on the nano-scale mechanical system and/or photocells while wearing laminated contact lens 100.

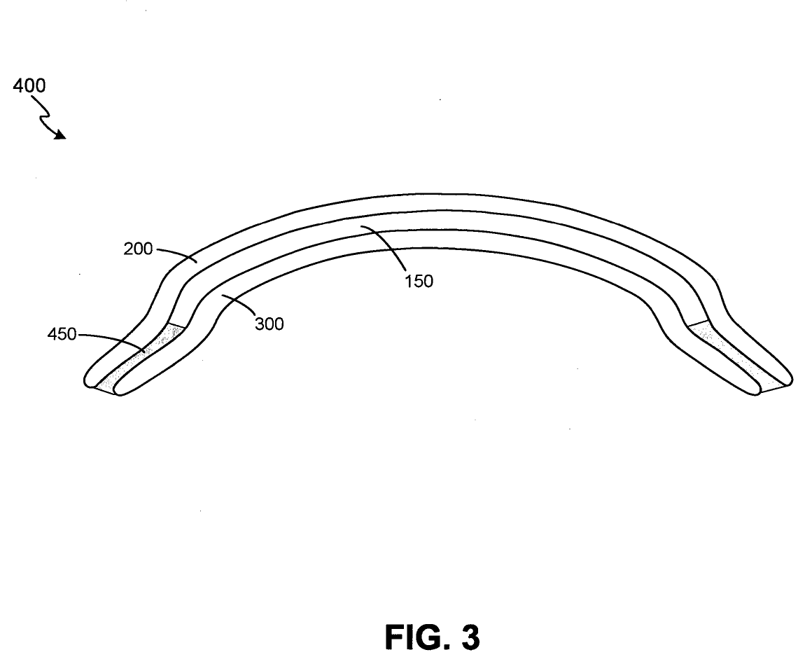

FIG. 3 is a diagram of another exemplary embodiment of a laminated contact lens 400 formed of two or more contact lenses coupled to one another, and including a bonding composition 450 capable of transporting one or more materials through bonding composition 450. Laminated contact lens 400, in various exemplary embodiments, includes contact lens 200 and contact lens 300 connected to one another, and forming chamber 150 similar to the embodiments discussed in FIGS. 1 and 2.

Bonding composition 450 may be any composition capable of bonding contact lens 200 to contact lens 300 and capable of allowing one or more materials (e.g., a medicament) to be transported through it. In one exemplary embodiment, bonding composition 450 is hydroxyethy methacrylate (HEMA). Furthermore, bonding composition 450 (and/or contact lenses 200, 300) is suitably configured such that the materials are transported in and/or out of chamber 150 at a pre-determined rate. In addition, laminated contact lens 400 may include any of the configurations/materials of any suitable embodiment discussed above and/or below.

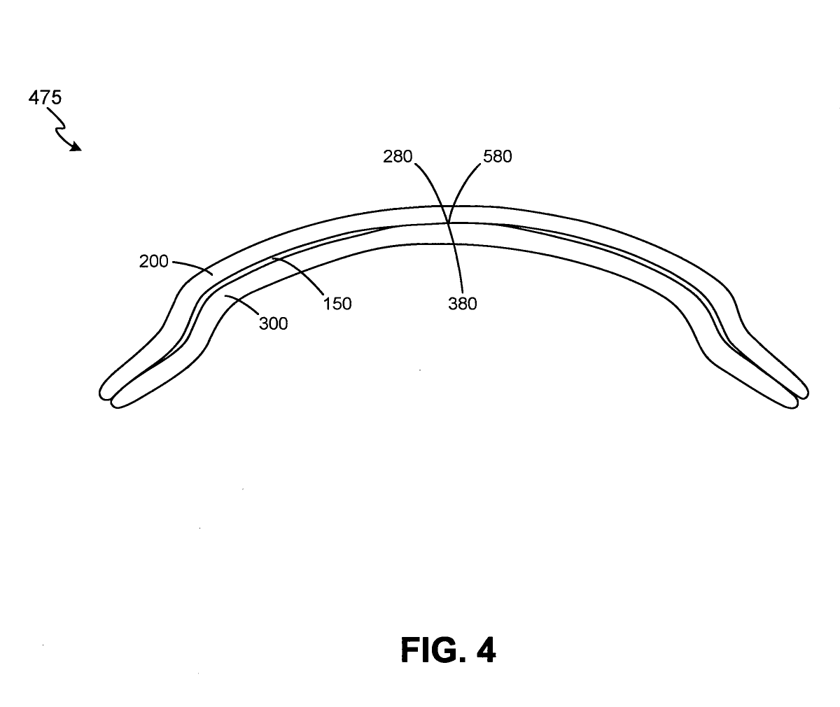

FIG. 4 is a diagram of yet another exemplary embodiment of a laminated contact lens 475 formed of two or more contact lenses coupled to one another. Laminated contact lens 475, in various exemplary embodiments, includes contact lens 200 and contact lens 300 connected to one another, and forming chamber 150 similar to the embodiments discussed in FIGS. 1, 2, and 3.

In one exemplary embodiment, contact lenses 200, 300 are further configured to contact each other at a central point 280 of contact lens 200 and a central point 380 of contact lens 300 to form central point 580 of contact lens 475. In accordance with an aspect of one exemplary embodiment of the invention, chamber 150 is filled with air or any other substance differing in refractive index from the lens material such that laminated contact lens 475 forms an aspheric bifocal contact lens, the benefits of which are well known in the art. Furthermore, laminated contact lens 475 may include any of the configurations/materials of any suitable embodiment discussed above.

Various exemplary embodiments of the invention also include a laminated contact lens having a plurality of chambers formed when two or more contact lenses are coupled to one another. In accordance with an aspect of these various exemplary embodiments, the central optic zone in each of the individual contact lenses are parallel to and/or make contact with one or more adjacent lenses. Such a configuration excludes gap components from the central optic zone of the laminated contact lens so that the plurality of lenses substantially do not interfere with light transmission, or if the presence of a gap in the center would create too much thickness, enables desirable oxygen transmission through the center of the laminated lens.

With reference now to FIG. 5, FIG. 5 is a diagram of a laminated contact lens 500 including a plurality of chambers in accordance with one exemplary embodiment of the invention. Laminated contact lens is formed by coupling a plurality of contact lens (e.g., contact lens 510 and contact lens 550) to one another. Notably, contact lenses 510 and 550 may be formed of the same materials and in a similar manner as contact lenses discussed above.

Contact lens 510 includes an exterior edge 515 and an interior edge 520. Furthermore, contact lens 510 includes a central optic zone 525. In accordance with one exemplary embodiment, central optic zone 525 is flatter than the cornea of a user of laminated contact lens 500. Similarly, contact lens 550 includes an exterior edge 555 and an interior edge 560. Furthermore, contact lens 550 includes a central optic zone 565. In accordance with one exemplary embodiment of the invention, central optic zones 525 and 565 are parallel to one another to form a central optic zone 575 when contact lens 510 is coupled to contact lens 550.

In accordance with one exemplary embodiment, exterior edge 515, interior edge 520, exterior edge 555, and interior edge 560 each include a curved shape. However, the PZD of contact lens 510 is different than the PZD of contact lens 550. As such, a plurality chambers (e.g., a chamber 530 and a chamber 535) are formed on each side of a central optic zone 575 by the gap created between interior edge 520 and exterior edge 555. Notably, chambers 530 and 535 are similar to chambers discussed above and may be empty or include the materials discussed above within one or both of chambers 530 and 535. Moreover, laminated contact lens 500 may includes holes in and/or gaps between the peripheral edges of contact lens 510 and 550 such that material with chambers 530 and/or 535 may come into contact with the eye of a user.

FIG. 6 is a diagram of a laminated contact lens 600 including a plurality of chambers similar to laminated contact lens 500 in accordance with an exemplary embodiment of the invention. Laminated contact lens 600 is formed by coupling a plurality of contact lens (e.g., contact lens 610 and contact lens 650) to one another. Furthermore, contact lens 600 includes a central optic zone 675 similar to central optic zone 575 discussed above. Moreover, contact lens 610 includes an exterior edge 615 and an interior edge 620, and contact lens 650 includes an exterior edge 655 and an interior edge 660.

In accordance with one exemplary embodiment, exterior edge 615 includes a sigmoidal shape, whereas interior edge 620, exterior edge 655, and interior edge 660 each include a curved shape. Similar to above, laminated contact lens 600 includes a chamber 630 and a chamber 635 formed by the difference between the PZD of contact lens 610 and the PZD of contact lens 650. In other words, chambers 630 and 635 are formed by the gap created between interior edge 620 and exterior edge 655.

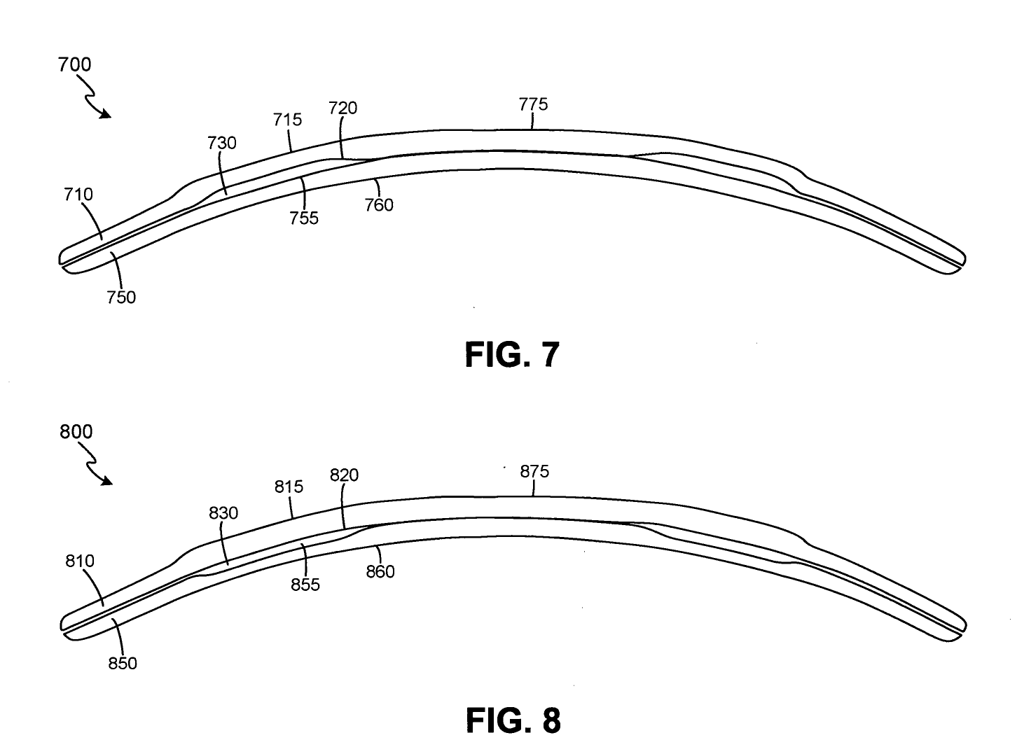

FIGS. 7-20 present various combinations of exemplary embodiments of a laminated contact lens including exterior edges and interior edges having either a curved shape or a sigmoidal shape. The changes in the curve cause changes in shape, which thus result in alteration of gap and lens thickness as desired based on a particular function or for comfort. Further, these curves determine which portions of the lenses are aligned so as to be in contact upon lamination. The individual analysis of each laminated contact lens embodiment is similar to laminated contact lenses 500 and 600 discussed above. For completeness, a brief description of each embodiment is presented below.

FIG. 7 is a diagram of a laminated contact lens 700 having a chamber 730, a central optic zone 775, and an exterior edge 715, an exterior edge 755, and an interior edge 760 including a curved shape, whereas an interior edge 720 includes a sigmoidal shape. FIG. 8 is a diagram of a laminated contact lens 800 having a chamber 830, a central optic zone 875, and an exterior edge 815, an interior edge 820, and an interior edge 860 including a curved shape, whereas an exterior edge 855 includes a sigmoidal shape.

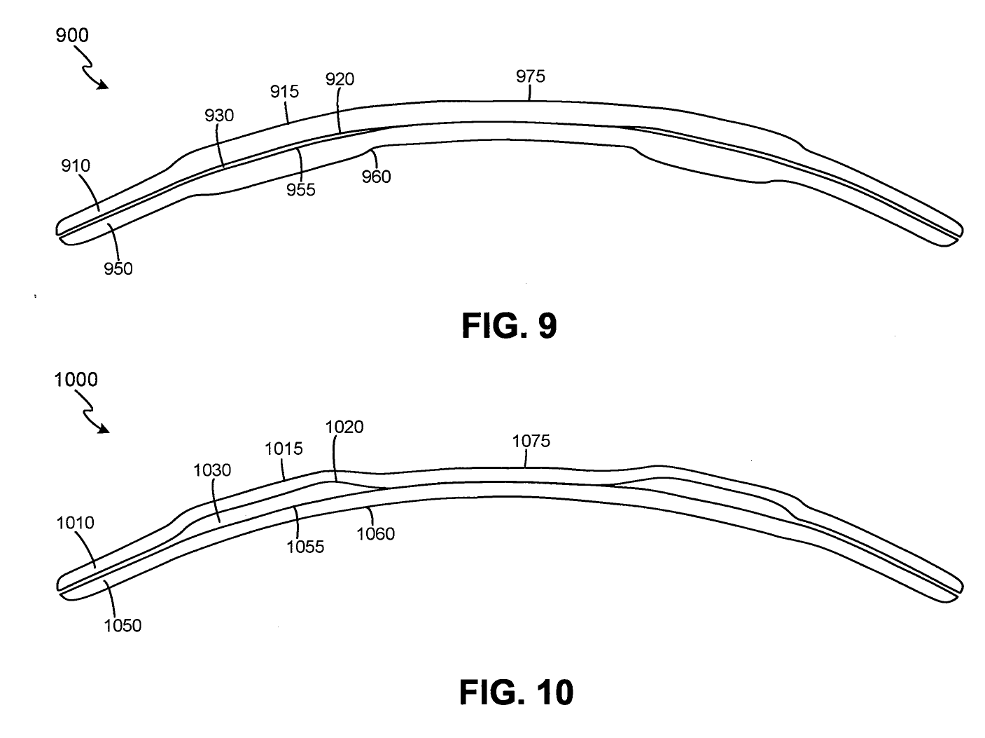

Laminated contact lens 900, as illustrated in FIG. 9, includes a chamber 930, a central optic zone 975, and an exterior edge 915, an interior edge 920, and an exterior edge 955 including a curved shape, whereas an interior edge 860 includes a sigmoidal shape. FIG. 10 is a diagram of a laminated contact lens 1000 having a chamber 1030, a central optic zone 1075, and an exterior edge 1055 and an interior edge 1060 including a curved shape, whereas an exterior edge 1015 and an interior edge 1020 include a sigmoidal shape.

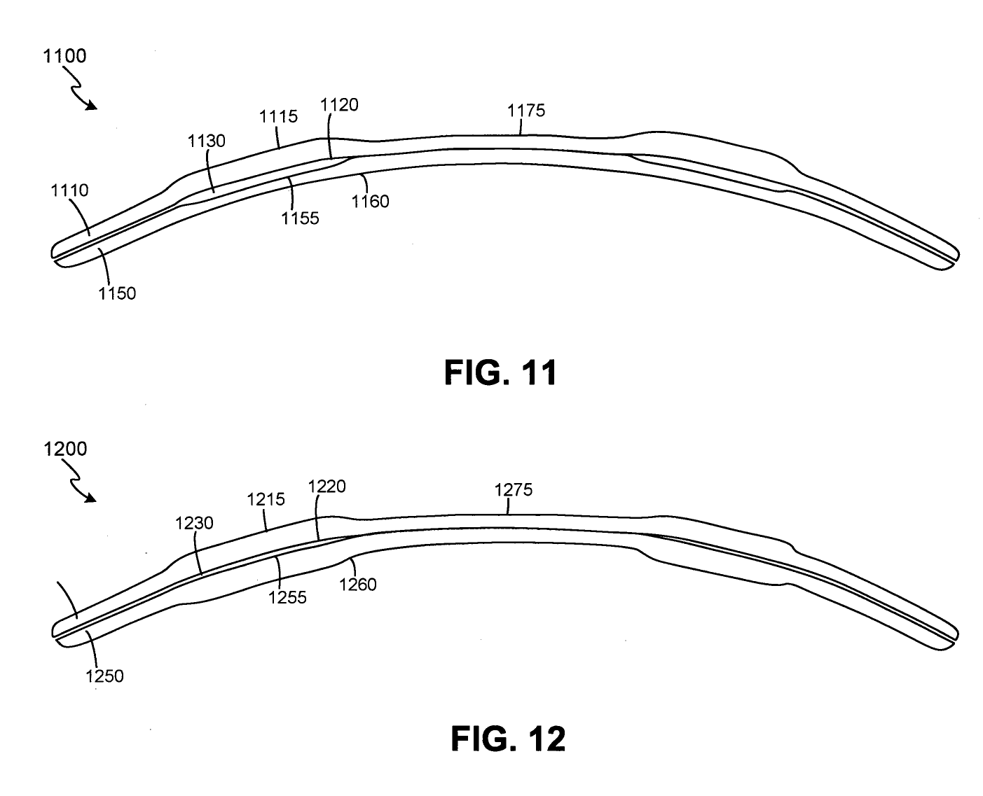

FIG. 11 is a diagram of a laminated contact lens 1100 having a chamber 1130, a central optic zone 1175, and an interior edge 1020 and an interior edge 1160 including a curved shape, whereas an exterior edge 1115 and an exterior edge 1155 include a sigmoidal shape. FIG. 12 is a diagram of a laminated contact lens 1200 having a chamber 1230, a central optic zone 1275, and an interior edge 1220 and an exterior edge 1255 including a curved shape, whereas an exterior edge 1215 and an interior edge 1260 include a sigmoidal shape.

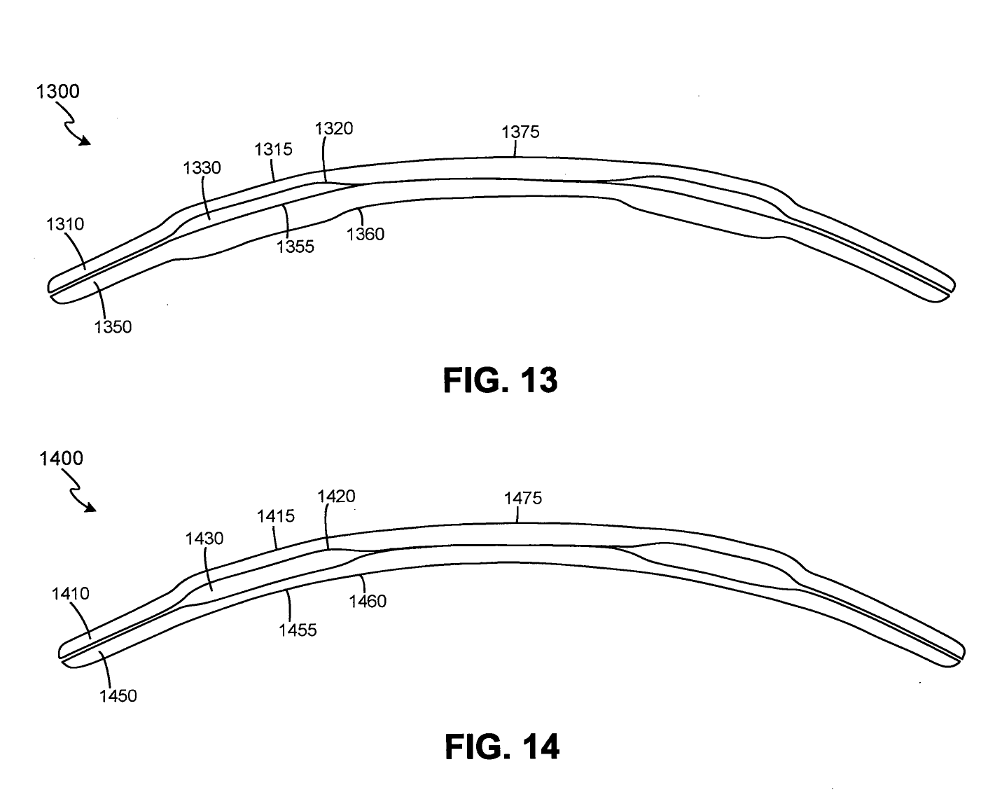

Laminated contact lens 1300, as shown in FIG. 13, includes a chamber 1330, a central optic zone 1375, and an exterior edge 1315 and an exterior edge 1355 including a curved shape, whereas an interior edge 1320 and an interior edge 1360 include a sigmoidal shape. FIG. 14 is a diagram of a laminated contact lens 1400 having a chamber 1430, a central optic zone 1475, and an exterior edge 1415 and an interior edge 1460 including a curved shape, whereas an interior edge 1420 and an exterior edge 1455 include a sigmoidal shape.

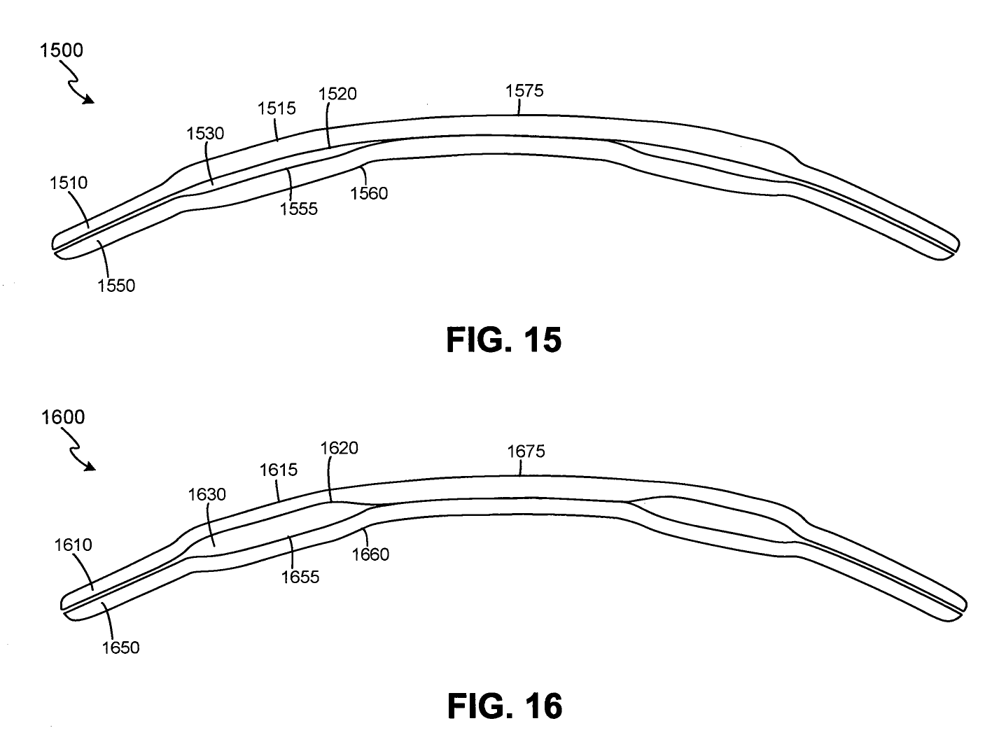

FIG. 15 is a diagram of a laminated contact lens 1500 having a chamber 1530, a central optic zone 1575, and an exterior edge 1515 and an interior edge 1520 including a curved shape, whereas an exterior edge 1555 and an interior edge 1560 include a sigmoidal shape. FIG. 16 is a diagram of a laminated contact lens 1600 having a chamber 1630, a central optic zone 1675, and an exterior edge 1615 including a curved shape, whereas an interior edge 1620, an exterior edge 1655, and an interior edge 1660 include a sigmoidal shape.

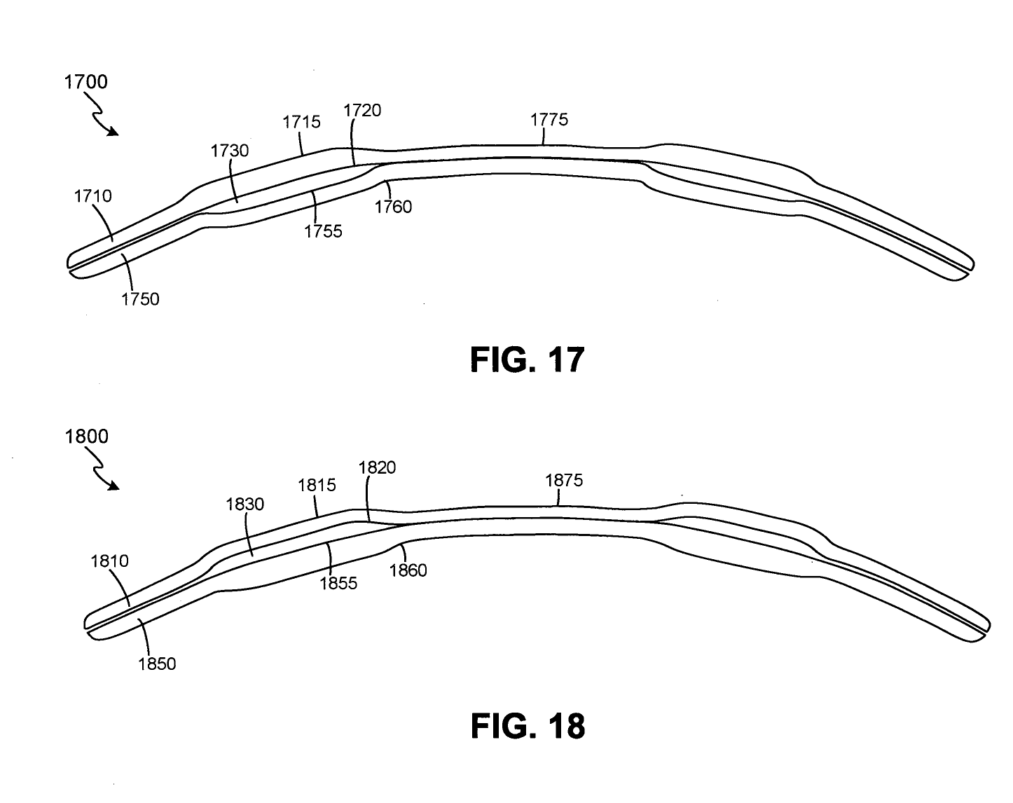

Laminated contact lens 1700, as illustrated in FIG. 17, includes a chamber 1730, a central optic zone 1775, and an interior edge 1620 including a curved shape, whereas an exterior edge 1715, an exterior edge 1755, and an interior edge 1760 include a sigmoidal shape. FIG. 18 is a diagram of a laminated contact lens 1800 having a chamber 1830, a central optic zone 1875, and an exterior edge 1855 including a curved shape, whereas an exterior edge 1815, an interior edge 1820, and an interior edge 1860 include a sigmoidal shape.

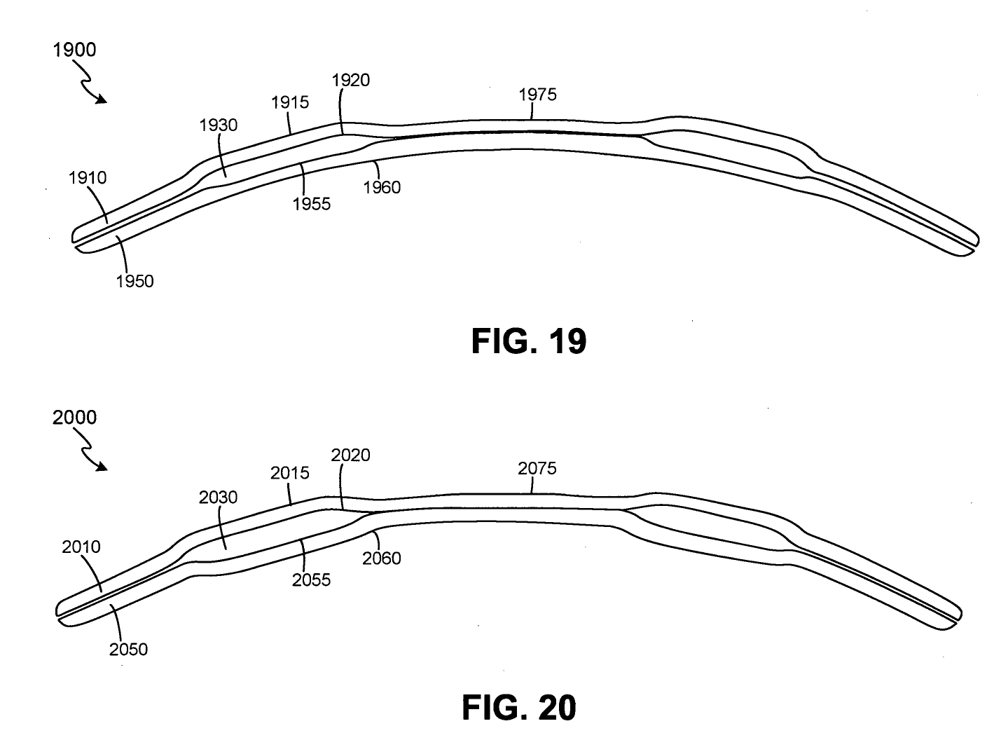

FIG. 19 is a diagram of a laminated contact lens 1900 having a chamber 1930, a central optic zone 1975, and an interior edge 1960 including a curved shape, whereas an exterior edge 1915, an interior edge 1920, and an exterior edge 1955 include a sigmoidal shape. FIG. 20 is a diagram of a laminated contact lens 2000 having a chamber 2030, a central optic zone 2075, and an exterior edge 2015, an interior edge 2020, an exterior edge 2055, and an interior edge 2060 each including a sigmoidal shape.

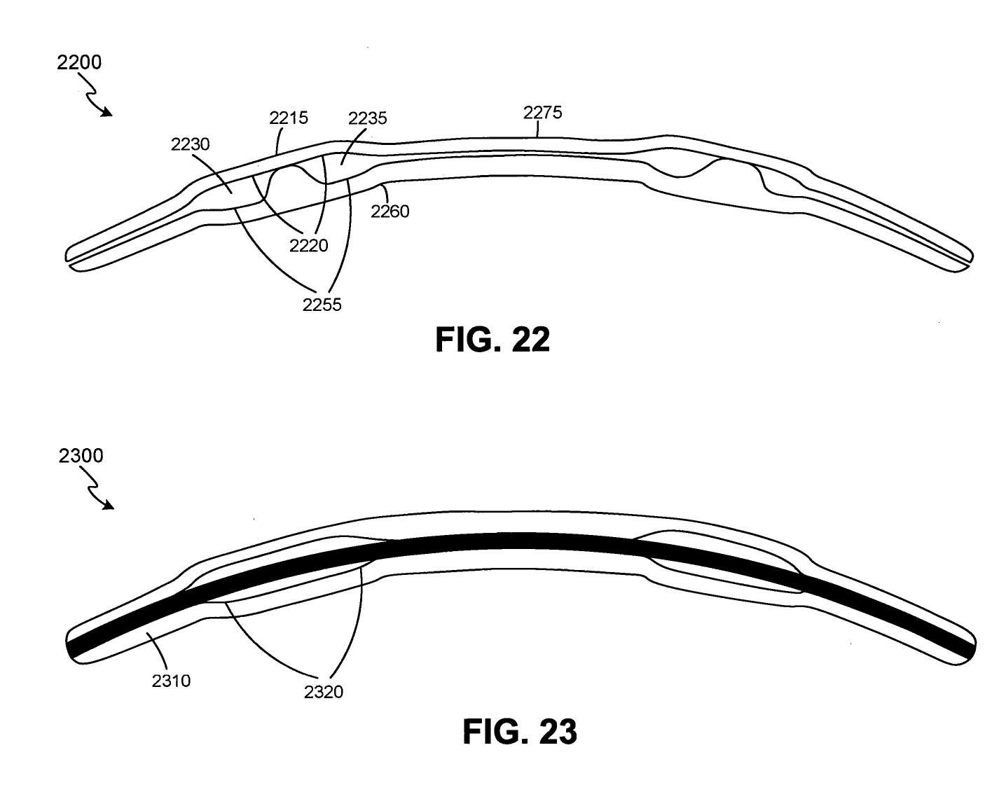

In contrast, FIG. 22 is a diagram of a laminated contact lens 2200 having a plurality of chambers 2230, 2235, a central optic zone 2275 and an interior edge 2260 including a curved shape, whereas an exterior edge 2215, an interior edge 2220 and an exterior edge 2255 include a sigmoidal shape. FIG. 23 is a diagram of a laminated contact lens 2300 shows an embodiment having a posterior lens 2310 with a plurality of sigmoid curves 2320 on its anterior surface, resulting in a secondary zone of contact with the anterior lens creating a second chamber not necessarily communicating with a first chamber.

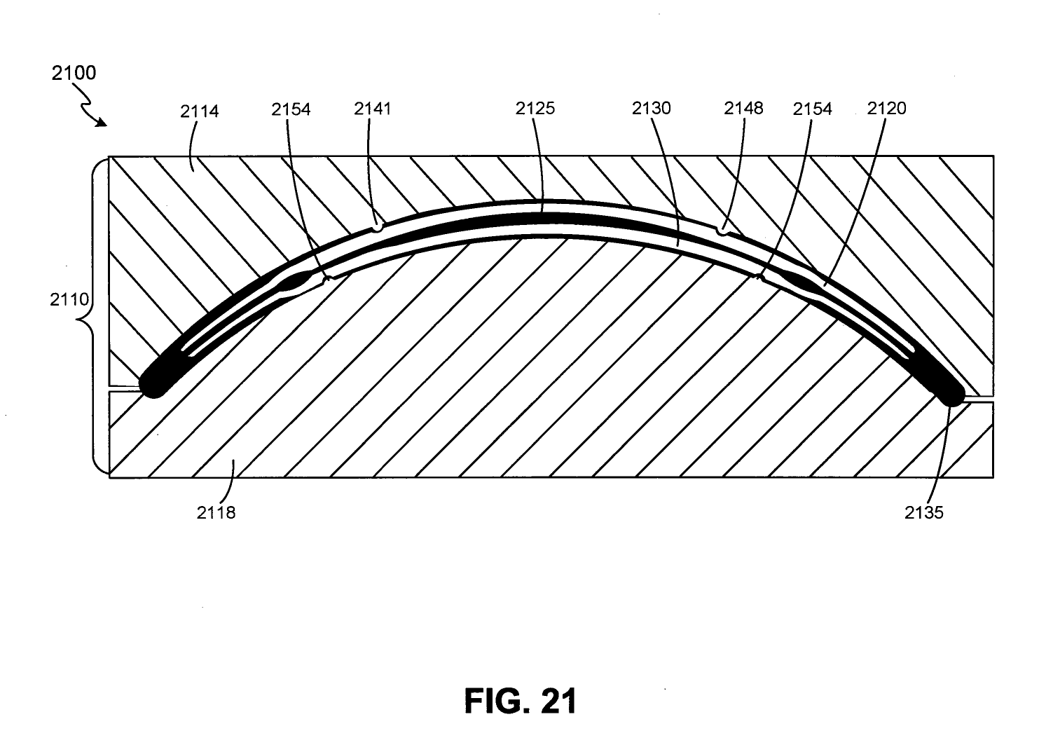

The following example discloses a method for producing a laminated contact lens 2100 in accordance with one exemplary embodiment of the invention. One embodiment includes production of laminated contact lens 2100 via a molding technique. As shown in FIG. 21, a two-piece mold 2110 is provided, wherein a first contact lens 2120 is inserted into an upper half 2114 of mold 2110, and a second contact lens 2130 is inserted into a lower half 2118 of mold 2110 before filling the mold with a polymerizable monomer 2125. Notably, each of contact lenses 2120 and 2130 is made prior to insertion into mold 2110.

Prior to closing mold 2110, monomer 2125 (which includes different mechanical and/or optical properties) is injected into mold 2110 between contact lenses 2120 and 2130. When exposed to initiating conditions, monomer 2125 polymerizes. Upon opening, mold 2110 yields laminated lens 2100 with a skirt 2135 having different properties (mechanical and/or optical) than each of contact lenses 2120 and 2130. In other words, laminated lens 2100 includes different properties in the center than contact lenses 2120 and 2130 and/or a cast component at its edge.

Notably, notches 2144 and 2148 on the cross section of the lens surface are reference indicators for centering contact lenses 2120 and 2130 in mold 2110 prior to filling it with monomer 2125. Preferably, the depth of notches 2144 and 2148 are kept to a minimum to prevent any adverse affects from occurring to laminated contact lens 2100. In addition, bulges 2154 and 2158 may be utilized to insure against the skirt polymer being pulled from between the contact lenses 2120 and 2130. However, if there is sufficient adhesion of contact lens 2120 to contact lens 2130, bulges 2154 and/or 2158 may not be needed.

The optical power of laminated contact lens 2100 is determined by the four curves in which light passes through on its way to the user's eye. Having the type of configuration discussed above reduces the number of molds 2100 needed to produce the needed range of lens powers, where typically such a laminated contact lens would include a rigid center and a soft skirt. Thus, laminated contact lens 2100 may be manufactured in an efficient manner. Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of the invention. All structural, chemical, and functional equivalents to the elements of the above-described exemplary embodiments that are known to those of ordinary skill in the art are expressly incorporated herein by reference and are intended to be encompassed by the invention.

We claim:

1. A laminated contact lens, comprising:
a first contact lens;
a second contact lens coupled to said first contact lens; and
a chamber formed between said first contact lens and said second contact lens.

2. The laminated contact lens of claim 1, wherein said first contact lens includes a first proximity zone depth (PZD) and said second contact lens includes a second PZD, wherein said first PZD and said second PZD are different proximity zone depths.

3. The laminated contact lens of claim 2, wherein said chamber is formed by a difference between said first PZD and said second PZD.

4. The laminated contact lens of claim 1, wherein said first contact lens is comprised of a hydrophilic monomer and said second contact lens is comprised of a hydrophilic monomer.

5. The laminated contact lens of claim 1, wherein said first contact lens is comprised of a hydrophobic monomer and said second contact lens is comprised of a hydrophobic monomer.

6. The laminated contact lens of claim 1, wherein said first contact lens is comprised of a hydrophobic monomer and said second contact lens is comprised of a hydrophilic monomer.

7. The laminated contact lens of claim 1, wherein the laminated contact lens includes a radius in the range of about 4.0 millimeters (mm) to about 8.0 mm.

8. The laminated contact lens of claim 1, further comprising:
a medicament housed within said chamber.

9. The laminated contact lens of claim 8, further comprising:
a bonding composition coupling said first contact lens to said second contact lens.

10. The laminated contact lens of claim 9, wherein said bonding composition is hydroxyethy methacrylate (HEMA).

11. The laminated contact lens of claim 10, wherein said HEMA is configured to transport said medicament at least one into and out of an eye of a user.

12. The laminated contact lens of claim 10, wherein said HEMA is configured to transport said medicament at least one into and out of an eye of a user at a pre-determined rate.

13. The laminated contact lens of claim 8, further comprising:
at least one hole in at least one of said first contact lens and said second contact lens such that said medicament is transported at least one of into and out of an eye of a user.

14. The laminated contact lens of claim 8, further comprising:
at least one hole in at least one of said first contact lens and said second contact lens such that said medicament is transported at least one of into and out of an eye of a user at a pre-determined rate.

15. The laminated contact lens of claim 8, further comprising:
a gap formed between a peripheral landing formed by a first periphery of said first contact lens and a second peripheral of said second contact lens such that said medicament is transported at least one of into and out of an eye of a user.

16. The laminated contact lens of claim 8, further comprising:
a gap formed between a peripheral landing formed by a first periphery of said first contact lens and a second peripheral of said second contact lens such that said medicament is transported at least one of into and out of an eye of a user at a pre-determined rate.

17. The laminated contact lens of claim 1, further comprising:
at least one of a gas, a liquid, and a solid housed within said chamber to improve the vision of a user.

18. The laminated contact lens of claim 1, further comprising:
a color component housed within said chamber to give the appearance that an eye of a user has a particular color.

19. The laminated contact lens of claim 1, further comprising:
at least one nano-scale mechanical system and at least one photocell housed in said chamber.

20. The laminated contact lens of claim 19, wherein said nano-scale mechanical system comprises:
communication media configured to display at least one of images and messages to a user.

21. The laminated contact lens of claim 1, wherein the lens has an invaginated edge.

22. The laminated contact lens of claim 1, wherein the lens has a semi-rounded edge.

23. A laminated contact lens, comprising:
a first contact lens;
a second contact lens coupled to said first contact lens;
a first chamber formed between said first contact lens and said second contact lens; and
a second chamber formed between said first contact lens and said second contact lens.

24. The laminated contact lens of claim 23, wherein said first contact lens includes a first proximity zone depth (PZD) and said second contact lens includes a second PZD, wherein said first PZD and said second PZD are different proximity zone depths.

25. The laminated contact lens of claim 24, wherein said first chamber and said second chamber are formed by a difference between said first PZD and said second PZD.

26. The laminated contact lens of claim 23, wherein:
said first contact lens includes a first exterior edge and a first interior edge; and
said second contact lens includes a second exterior edge and a second interior edge.

27. The laminated contact lens of claim 26, wherein:
at least one said first exterior edge, said first interior edge, said second exterior edge, and said second interior edge includes a sigmoidal shape, with the remaining edges including a curved shape.

28. The laminated contact lens of claim 26, wherein:
each of said first exterior edge, said first interior edge, said second exterior edge, and said second interior edge includes a sigmoidal shape.

29. The laminated contact lens of claim 26, wherein:
each of said first exterior edge, said first interior edge, said second exterior edge, and said second interior edge includes a curved shape.
